(19) United States
(12) Patent Application Publication
(10) Pub. No.: US 2006/0025364 A1
(43) Pub. Date: Feb. 2, 2006

(54) Regulation of peritoneal healing and adhesion development

(76) Inventors: Michael P. Diamond, Grosse Pointe Shores, MI (US); Ghassan M. Saed, West Bloomfield, MI (US)

Correspondence Address: FISH & NEAVE IP GROUP;ROPES & GRAY LLP, ONE INTERNATIONAL PLACE, BOSTON, MA, 02110-2624, US

(21) Appl. No.: 11/081,278

(22) Filed: Mar. 16, 2005

Related U.S. Application Data
(60) Provisional application No. 60/554,275, filed on Mar. 18, 2004.

(51) Int. Cl. A61K 48/00

(52) U.S. Cl. 514/44

(57) ABSTRACT

Methods for the prevention of adhesion formation and development, and for the stimulation of fibrosis, involve the administration of therapeutic formulations to a patient containing inhibitors or stimulators to selected molecular adhesion markers. The molecular markers of the invention include Caspase 2, Caspase 3, Caspase 9, PPARα, PPARβ, PPARγ1, PPARγ2, and NF-kappa B.

### CROSS-REFERENCE TO RELATED APPLICATIONS

This Application claims priority to U.S. Provisional Patent Application No. 60/554,275, filed on Mar. 18, 2004, the disclosure of which is incorporated herein in its entirety by reference.

### BACKGROUND OF THE INVENTION

The peritoneum overlying the organs and cavity walls within the abdomen is composed of mesothelial cells, extracellular matrix material, fibroblasts, macrophages, endothelial cells, muscle cells, monocytes and blood vessels. Injuries to the peritoneal surfaces of the peritoneum, whether due to infection, endometriosis, pathological processes, or tissue trauma induced during surgical procedures, result in the development of post-operative adhesions in the vast majority of patients.

The cellular processes resulting in either normal peritoneal tissue repair, or the development of adhesions, include migration, proliferation, apoptosis and/or differentiation of several cell types, among them inflammatory, immune, mesothelial and fibroblast cells. Molecules produced locally by these cells regulate fibrinolytic activity, tissue remodeling and angiogenesis, as well as the synthesis and deposition of extracellular matrix material (ECM), and these processes are all central to the development of adhesions.

The molecular events underlying peritoneal wound healing and the development of fibrous adhesions are complex, multifaceted and not well defined. While many phases of peritoneal wound repair, and specific mechanisms that regulate their cellular activities, resemble dermal wound healing, two fundamental differences exist.

First, in contrast to dermal injuries, which heal from the edges toward the middle (such that the time of healing is dependent on the length of the lesion), peritoneal injuries are thought to heal by differentiation of the underlying progenitor cells (such that healing occurs throughout the lesion simultaneously, independent of the surface area of the injury).

Second, peritoneal wounds are continuously exposed to factors present in the peritoneal fluid, such as growth factors and cytokines. Many of these substances are synthesized and released by mesothelial cells, activated macrophages, and various cells within the wound. Therefore, an autocrine/paracrine feedback is an important component of peritoneal healing. For normal peritoneal healing to occur, the availability of these signaling substances must be optimal, precise and synchronized. Inhibition, interruption, or excessive expression of these signals may be responsible for a failure in normal healing, leading either to impairment (non-healing) or excessive formation of scar tissue (adhesions).

The increased incidence of postoperative adhesions and their complications have refocused attention on adhesion development, its clinical consequences and its prevention. Although the understanding of the pathogenesis of adhesions has improved in recent years, the molecular mechanisms involved continue to be delineated by the scientific community.

Adhesions generally result from the normal peritoneal wound healing response and develop in the first 5-7 days after the injury. Postsurgical peritoneal repair begins with blood coagulation, which releases a variety of chemical messengers that initiate a cascade of events. Some of the principal cellular elements in this cascade are leukocytes, including polymorphonuclear neutrophils and macrophages, mesothelial cells, fibroblast cells, and fibrin. Following surgical injury, macrophages exhibit increased phagocytic, respiratory burst and secretory activity, and after about 5 days, are the major components of the leukocyte population. Macrophages also recruit new mesothelial and fibroblast cells to the surface of the injury. These cells form small islands throughout the injured area, and proliferate into sheets of mesothelial cells. After about 3-5 days following the surgical injury, re-epithelialization of the injured area occurs.

PCT application no. WO 00/20642, published Apr. 13, 2000, discloses methods for the prevention of adhesion development by the administration of therapeutic formulations containing TIMP-1 inhibitors to a patient. TIMP-1 is part of a family of inhibitors of metalloproteinase proteins, or TIMP's, which regulate the catalytic activity of matrix metalloproteinases (MMP's). The administration of TIMP-1 inhibitors to a patient is believed to alter the local levels of both TIMP-1 and MMP, and specifically to reduce the expression of TIMP-1, to thereby inhibit the development of adhesions.

PCT application no. PCT/US02/07119, filed Mar. 11, 2002, discloses methods for preventing adhesion formation and development by administering a therapeutic formulation to a patient in need of treatment to modulate the rate of apoptosis in fibroblast adhesion cells. These formulations can include, as active ingredients, the protein Bax, Bax enhancers, such as p53, Bax antagonists, Bcl-2 protein inhibitors and Bcl-2 protein antagonists. PCT application no. PCT/US02/07119 also includes a method for determining the predisposition of a subject to adhesion formation by measuring the Bcl-2/Bax ratio at multiple sites within the subject.

PCT application no. PCT/US02/07290, filed Mar. 11, 2002, discloses methods for preventing adhesion formation and development by the administration of therapeutic formulations to a patient which include, as active ingredients, IFN-γ and IFN-γ protein enhancers. The IFN-γ and IFN-γ enhancers can be administered directly to the fibrosis and/or healing tissue of a patient in need of treatment.

The respective disclosures of each of the above-identified PCT patent applications is incorporated herein in its entirety by reference.

It has been suggested that peritoneal adhesions develop in the vast majority of subjects, with more frequent occurrence in certain subjects following surgical procedures to the exclusion of other unaffected subjects, and at particular sites within the affected subject. The molecular basis for such predisposition is not known.

Accordingly, it is an objective of this invention to provide a method for treating, preventing and reducing the incidence of post-operative surgical adhesions in subjects. It is a further objective of this invention to identify suitable molecular markers for adhesion development, and to propose treatment methods incorporating stimulators or inhibitors for remediating the development of adhesions in subjects. It is a still further objective of the invention to provide therapeutic formulations capable of stimulating the fibrosis of certain tissues in instances where it may be necessary or desirable to adhere certain organs or tissue surfaces to other surfaces within the body of a subject.

### SUMMARY OF THE INVENTION

Molecular markers of adhesion development have been identified, and methods for using inhibitors or enhancers of these markers to treat and prevent adhesions in certain subjects are provided. The regulation of these molecular markers can significantly impact the development of adhesions in the body of a subject.

Accordingly, in one embodiment, the invention comprises inhibitors of molecular markers of adhesion development. The inhibitors prevent or retard the development of adhesions in subjects following a surgical event.

The molecular markers of this invention include certain members of the caspase family of proteases, and specifically Caspase 2, Caspase 3 and Caspase 9; certain members of the PPAR (Peroxisome Proliferator-Activated Receptor) family, namely PPARα, PPARβ, PPARγ1 and PPARγ2; and NF-kappa B.

In a further embodiment, the invention comprises stimulators of the above molecular markers that can be used for stimulating fibrosis at selected sites within the body of a subject. The stimulation of fibrosis is useful where it is desired to cause adherence of selected tissues within the body, such as the adherence of an organ to a site within the body cavity to reposition the organ in order to ameliorate a preexisting medial condition.

In additional embodiments, pharmaceutical compositions which include the inhibitors or stimulators of surgical adhesions described herein, as well as suitable adjuvants, excipients and other additives, are also provided.

Although many such inhibitory and stimulatory substances are known to those skilled in the art, the present invention is intended to encompass both known inhibitors and stimulators, as well as inhibitors and stimulators that may be subsequently discovered and elucidated by the inventors and others.

### DETAILED DESCRIPTION OF THE INVENTION

Peritoneal mesothelial cells that line the serosal surface of the peritoneal cavity provide a natural protective barrier that prevents the organs from adhering to adjacent opposing surfaces. However, cellular or tissue injury induced following a surgical procedure, an infection or inflammation, compromises the integrity of the mesothelial cells, and can result in a local biological response with the objective of repairing the defective surfaces. If a cellular or tissue injury is relatively extensive, it can lead to excess migration and proliferation of various wound cells, such as fibroblasts. This response initiates a cascade of events that often result in the development of peritoneal adhesions, known to be a major cause of bowel obstruction, pain, infertility, and hospital readmissions.

Adhesion reformation is known to occur more frequently than de novo adhesion formation. Tissue remodeling during the wound healing process is governed by the dynamic equilibrium between cell growth and programmed cell death, or apoptosis. The control of cell growth and apoptosis are intimately associated, and a disturbance of the balance between these two processes often leads to pathological events, such as cell accumulations in cancer cells and tissue fibrosis.

It has now been found that certain molecular markers are expressed differently in fibroblasts obtained from normal peritoneum tissue and in fibroblasts obtained from adhesion tissue, and that this difference can be exploited to develop effective treatments for adhesions at the molecular level. This can be achieved by modifying the expression of these molecular markers to permit the regulation of tissue healing and tissue fibrosis, including the reduction of adhesion development, and the acceleration of fibrosis and scarring.

The mechanism by which inhibitory substances to these molecular markers impact the development of adhesions is not completely understood. However, it is known that fibroblasts are exposed to hypoxia during ischemic events, and that hypoxia inhibits apoptosis and enhances proliferation of peritoneum and adhesion fibroblast cells and tissue. By removing, or reversing, the effect of these inhibitory factors, it is believed that the rate of apoptosis of adhesion fibroblasts can be increased, and adhesion formation can be impeded.

As used herein, the following terms and phrases shall have the following general meanings, unless indicated otherwise.

By “inhibitor” is meant, in the context of this invention, a substance which is effective to inhibit, reduce, modify or block the level of expression of the molecular adhesion marker on fibroblast cells at the site of the potential adhesion. Suitable inhibitory substances include, inter alia, substances which are effective to inhibit the molecular markers of adhesion development at the protein expression level, such as antibodies and antagonists; substances which reduce posttranslational modifications, such as glycosylation or methylation; substances effective to inhibit activity at the mRNA translation level, such as antisense oligonucleotides; and substances effective to inhibit the activity at the gene transcription level, such as oligonucleotides that bind to the gene promoter region of the protein.

A “stimulator” or “stimulatory substance” has the opposite effect of an inhibitor or inhibitory substance. Suitable stimulatory substances include, for instance, agonists effective at the protein level, and enhancers effective at the gene level.

The types of inhibitors or stimulators used in the present invention can vary by their physical characteristics and chemical structure. Typical molecules include soluble forms of the molecular markers of the invention, or fragments thereof, inhibitory proteins, such as antibodies, and particularly monoclonal antibodies, humanized antibodies, chimeric antibodies, polyclonal antibodies, and Fab₂ fragments; inhibitory peptides, including protein fragments; chemical entities; small molecules; and short interfering RNAs that downregulate the expression of the markers. Short interfering RNAs are generally known in the art. See, for example, T. R. Brummelkamp et al., Science, 296, pages 550-553 (2002), the disclosure of which is incorporated herein in its entirety.

The terms “treat” or “treatment” are intended to include both prophylactic (vaccines) and therapeutic treatments, and generally denote the administration of a therapeutic agent to a subject having a disease or disorder, a symptom of a disease or disorder, or a predisposition toward a disease or disorder, for the purpose of preventing alleviating, relieving, reducing the symptoms of, altering, or improving the medical condition or disorder. The methods of treatment described herein may be specifically modified or tailored based on a specific knowledge of the subject obtained by pharmacogenomics, and other methods for analyzing individual drug responses to therapies.

A “therapeutically effective amount” of a pharmaceutical composition means that amount which is capable of treating, preventing, or reversing the symptoms of the medical condition or disease. A therapeutically effective amount can be determined on an individual basis and is based, at least in part, on a consideration of the particular species of mammal, for example, the mammal's size, the particular inhibitor or stimulator used, the type of delivery system used, and the time of administration relative to the progression of the disease. A therapeutically effective amount can be determined by one of ordinary skill in the art employing such factors and using no more than routine experimentation.

The terms “prevent” and “preventing” as used herein refer to completely or partially inhibiting a biological response, as well as inhibiting an increase in a biological response. For instance, the prevention of adhesion development refers to partially or completely inhibiting adhesion formation and adhesion reformation, as well as inhibiting an increase in adhesion formation and adhesion reformation.

The term “subject,” as used herein, means a human or non-human mammal, including but not limited to, a dog, cat, horse, cow, pig, sheep, goat, chicken, primate, rat, and mouse.

“Hypoxia” can be defined as a lack of oxygen to the cells and tissues, and this can disrupt the aerobic metabolism and synthesis of adenosine triphosphate (ATP) in cells, and hence the survival of the cells. Hypoxia can be simulated experimentally by limiting the amount of oxygen to the cells or tissue to, for instance, a 2% oxygen blanket in a controlled environment.

The adhesion markers of the present invention include the following molecules: Caspase 2, Caspase 3 and Caspase 9, PPARα, PPARβ, PPARγ1 and PPARγ2; and NF-kappa B, as well as mixtures of the foregoing molecules.

The caspases are a family of proteases present in cells as latent enzymes. One subgroup of caspases is involved in apoptosis. A second subgroup of caspases is involved in processing a select group of cytokines, such as IL-1β and IL-1α. Caspases are thiol-proteases that cleave the carboxy terminal of aspirate residues. The caspases which are useful in the practice of this invention are, in particular, Caspase 2, Caspase 3 and Caspase 9.

The peroxisome proliferator-activated receptors (PPAR) are ligand-activated transcription factors that are related to retinoid, steroid and hormone receptors. PPARs are known to play a role in metabolism, cell proliferation, differentiation, adipogenesis and inflammatory signaling. The PPARs which are useful in the practice of this invention include PPARα, PPARβ, PPARγ1 and PPARγ2.

NF-kappa B protein is a transcription factor that is activated by proinflammatory signals or the engagement of Ag receptors.

Suitable inhibitors and stimulators of the molecular markers identified above can be formulated into compositions for the treatment of adhesions as described in more detail herein. The inhibitors of the invention include substances which are effective to inhibit or block the level of expression of the molecular marker on fibroblast cells at the site of the potential adhesion. Suitable inhibitory substances include substances effective to inhibit the molecule at the protein expression level, such as antibodies and antagonists; substances effective to inhibit activity at the mRNA translation level, such as antisense oligonucleotides; and substances effective to inhibit activity at the gene transcription level, such as oligonucleotides that bind to the gene promoter region. Although many such inhibitory substances are known to those skilled in the art, the invention is intended to encompass both known inhibitors, as well as inhibitors that may be subsequently discovered and elucidated.

Inhibitors designed to operate at the protein level by a post-translational mechanism include those inhibitors capable of both competitive and non-competitive inhibition. These inhibitors include molecules capable of competing with binding sites on the protein, or those capable of inactivating the protein, and include specifically antagonists, antibodies and inhibitory ligands.

Inhibitors designed to function at the mRNA level by a translational mechanism include antisense molecules, which function to prevent the translation of a protein from its specific mRNA, and agents that regulate the stability of mRNA.

Inhibitors which function at the gene level by a transcriptional mechanism generally involve the use of specific proteins and/or agents that bind to promoter regions of the gene, and prevent trans-acting elements from enhancing the transcription of the gene.

A stimulator has the opposite effect of an inhibitor. Such effects include the stimulation of fibrosis development at selected tissues in the body at selected sites. Typical stimulatory substances include protein agonists and gene enhancers. The stimulation of fibrosis is useful when it is desired to cause adherence of selected tissues within the body to each other, such as the adherence of an organ to a site within the body cavity to reposition the organ in order to ameliorate a preexisting medical condition.

The types of tissues that can be successfully treated according to the foregoing treatment procedures include peritoneal tissue, pleura tissue, pericardium tissue, ligaments, tendons, nerve sheaths, muscles and synovial tissue. Adhesion formation in such tissue typically occurs following, and as a result of, a surgical procedure, but can also occur during the healing of other acute and chronic pathologic processes.

The administration of the preparations of the invention to potentially affected tissue and organs, locally or systemically, can induce protection against postoperative surgical adhesion development, or adhesion reformation. The preparations of the invention are useful for treating or preventing adhesions that form at a site and that have potential or actual deleterious effects.

The following sites in the body can be successfully treated according to the method of this invention, and include, but are not limited to: the abdominal cavity, including intestine to intestine, and intestine to peritoneum; the pelvic cavity, including adhesions of the uterus, ovaries or fallopian tubes to other structures including each other and the pelvic wall; tendons and their support structures, including tendon to synovium; the repair of nerve sheaths; the repair of the spinal column or disks; the pericardium; the treatment of joints for inflammation, muscles, lung to chest wall adhesion, and to prevent pannus formation; the extraoccular muscle, to prevent adhesions from limiting the field of vision; and any situation in which adhesions form and impair function or cause pain.

The prevention of postoperative surgical adhesion development in a subject includes prophylactic treatment to prevent adhesion development following planned or elective surgical procedures, as well as following emergency operations.

In addition to the surgical procedures described above, elective surgeries within the scope of this invention include the following intraabdominal surgeries: right hemicolectomy; left hemicolectomy; ovarian cystectomy, sigmoid colectomy; tuboplasty; subtotal colectomy; total colectomy; laparoscopic or open cholecystectomy; hysterectomy, oophorectomy, salpingectomy, spinal disectomy; adheseolyses; myomectomy; cesarean section; tubal ligation; treatment of endometriosis, treatment of ectopic pregnancy, gastrectomy; pancreatectomy; splenectomy; liver, pancreas, small bowel, or kidney transplantation; lysis of adhesions; cesarean sections and other pelvic procedures, uterine surgery, etc.

Emergency intraabdominal surgeries within the scope of this invention include those surgeries used to correct the following conditions: perforated ulcer (duodenal or gastric); perforated diverticulitis; obstructive diverticulitis; bowel obstruction; perforated appendicitis; blunt abdominal trauma; eye surgeries; penetrating abdominal trauma; ruptured abdominal aortic aneurysm, cardiac surgeries, ectopic pregnancy; open and endoscopic orthopedic surgeries, neurosurgeries, gynecologic and pelvic surgeries, and surgeries to correct wound infections.

The preparations of this invention can be administered to a subject in an effective amount for inducing protection against postoperative surgical adhesion development. An “effective amount” for inducing protection against postoperative surgical adhesion development, as used herein, is that amount of pharmaceutical composition that will, alone or together with further doses or additional therapeutic compounds, inhibit or prevent the development of postoperative surgical adhesions.

The preparations of the invention when administered “in conjunction with” a surgical procedure, are administered close enough in time with the surgery or trauma that predispose the host to adhesion development, so that a protective effect against the particular disorder is obtained. The preparations may be administered long before the surgery, e.g., in the case of elective surgery (i.e., weeks or even months), preferably with booster administrations closer in time to (and even after) the surgery. Particularly in emergency situations, the preparations may be administered immediately before (minutes to hours), during and/or after the surgery. It is important only that the preparation are administered close enough in time so as to enhance the subject's response against adhesions, thereby increasing the chances of a successful host response and reducing the likelihood of adhesion development.

The present invention provides pharmaceutical compositions for medical use, which in some aspects comprise the preparations of the invention together with one or more pharmaceutically acceptable carriers and optionally other therapeutic ingredients. Thus the invention may also include pharmaceutical compositions in combination with an anti-infectious agent such as an antibacterial or anti-viral agent, an anti-inflammatory agent, an antibiotic, or other therapeutic agent, and a pharmaceutically acceptable carrier. The pharmaceutical compositions useful in the invention may be delivered separately with the other therapeutic agents, or in the form of therapeutic cocktails. A therapeutic cocktail includes a mixture of the pharmaceutical composition of the invention and another therapeutic agent. In this embodiment, a common administration vehicle (e.g., tablet, implant, injectable solution, etc.) contains both the pharmaceutical composition and another therapeutic agent. Alternatively, the other therapeutic agent can be separately dosed if desired. A barrier material, such as hyaluronic acid or carboxymethyl cellulose, can also be used as a carrier for the compositions of this invention.

The precise amount of the therapeutic agent used in combination with the pharmaceutical compositions of the invention depends upon a variety of factors, including the particular pharmaceutical composition selected, the dose and dose-timing selected, the mode of administration, the nature of any surgical or medical procedure contemplated, and the characteristics of the subject. Where local administration is carried out, it will be understood that very small amounts of the pharmaceutical composition may be required (nanograms and possibly picograms). The precise amounts selected can be determined without undue experimentation, particularly since a threshold amount is any amount which will favorably enhance the response.

Multiple doses of the pharmaceutical compositions of the invention are contemplated. For instance, when being administered in conjunction with a surgical procedure, the compositions of the invention can be administered in multiple doses over a three week to one day period preceding surgery. Further, doses may be administered post surgery as well. Any regimen that prevents or retards the development of adhesions may be used, although optimum doses and dosing regimens are those that not only inhibit the development of adhesion formation, but also result in protection against adhesion development. Desired time intervals for the delivery of multiple doses of a particular pharmaceutical composition can be determined by one of ordinary skill in the art employing no more than routine experimentation.

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

Suitable buffering agents include: acetic acid or its salt (1-2% w/v); citric acid or its salt (1-3% w/v); boric acid or its salt (0.5-2.5% w/v); succinic acid; and phosphoric acid or its salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The pharmaceutical compositions of the invention contain an effective amount of a pharmaceutical composition optionally included in a pharmaceutically acceptable carrier. The term “pharmaceutically acceptable carrier” means one or more compatible solid or liquid fillers, dilutants or encapsulating substances which are suitable for administration to a human or other animal. The term “carrier” denotes an organic or inorganic ingredient, natural or synthetic, with which the active ingredient is combined to facilitate the application of the composition to the subject. The components of the pharmaceutical compositions also are capable of being commingled with the pharmaceutical compositions of the present invention, and with each other, in a manner such that there is no interaction which would substantially impair the desired pharmaceutical efficiency.

Compositions suitable for parenteral administration conveniently comprise sterile aqueous preparations, which can be isotonic with the blood of the recipient. Among the acceptable vehicles and solvents are water, Ringer's solution, and isotonic sodium chloride solution. In addition, sterile, fixed oils are conventionally employed as a solvent or suspending medium. For this purpose, any bland fixed oil may be employed including synthetic mono-or di-glycerides. In addition, fatty acids such as oleic acid, find use in the preparation of injectables. Carrier formulations suitable for subcutaneous, intramuscular, intraperitoneal or intravenous administration may be found in Remington's Pharmaceutical Sciences, Mack Publishing Company, Easton, Pa.

The pharmaceutical compositions useful in the invention may be delivered in mixtures of more than one pharmaceutical composition. A mixture may consist of several pharmaceutical compositions.

A variety of administration routes are available. The particular mode selected will depend, of course, upon the particular pharmaceutical composition, the particular condition being treated, and the dosage required for therapeutic efficacy. The methods of this invention, generally speaking, may be practiced using any mode of administration that is medically acceptable, meaning any mode that produces effective levels of an immune response without causing clinically unacceptable adverse effects. Preferred modes of administration include parenteral, injection, infusion, deposition, implantation, anal or vaginal supposition, oral ingestion, inhalation, topical administration. Injections can be intravenous, intradermal, subcutaneous, intramuscular, or interperitoneal. For example, the pharmaceutical composition can be injected directly into the surgical site for the prevention of adhesions. In some embodiments, the injections can be given at multiple locations. Implantation includes inserting implantable drug delivery systems, e.g., microspheres, hydrogels, polymeric reservoirs, cholesterol matrixes, polymeric systems, e.g., matrix erosion and/or diffusion systems and non-polymeric systems, e.g., compressed, fused, or partially-fused pellets. Inhalation includes administering the pharmaceutical composition with an aerosol in an inhaler, either alone or attached to a carrier that can be absorbed. For systemic administration, it may be preferred that the pharmaceutical composition is encapsulated in liposomes. The term “parenteral” includes subcutaneous injections, intravenous, intramuscular, intraperitoneal, intrasternal injection or infusion techniques.

In certain preferred embodiments of the invention, the administration can be designed to result in the sequential exposure of the pharmaceutical composition over some period of time, e.g., hours, days, weeks, months or years. This can be accomplished by repeated administrations of the pharmaceutical composition, by one of the methods described above, or alternatively, by a sustained-release delivery system in which the pharmaceutical composition is delivered to the subject for a prolonged period without repeated administrations. By sustained-release delivery system is meant that the total release of the pharmaceutical composition does not occur immediately upon administration, but rather is delayed for some period of time. Release can occur in bursts, or it can occur gradually and continuously. Administration of such a system can be, e.g., by long-lasting oral dosage forms, bolus injections, transdermal patches, and subcutaneous implants.

Examples of systems in which release occurs in bursts includes, e.g., systems in which the pharmaceutical composition is entrapped in liposomes which are encapsulated in a polymer matrix, the liposomes being sensitive to specific stimuli, e.g., temperature, pH, light or a degrading enzyme, and systems in which the pharmaceutical composition is encapsulated by an ionically-coated microcapsule with a microcapsule core degrading enzyme. Examples of systems in which release of the pharmaceutical composition is gradual and continuous include, e.g., erosional systems in which the pharmaceutical composition is contained in a form within a matrix, and effusional systems in which the pharmaceutical composition permeates at a controlled rate, e.g., through a polymer. Such sustained release systems can be e.g., in the form of pellets, or capsules.

Both non-biodegradable and biodegradable polymeric matrices can be used to deliver the pharmaceutical compositions to the subject. Biodegradable matrices are preferred. Such polymers may be natural or synthetic polymers. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water, and further optionally is cross-linked with multi-valent ions or other polymers.

Bioadhesive polymers of particular interest include bioerodible hydrogels described by H. S. Sawhney, C. P. Pathak and J. A. Hubell in Macromolecules, (1993) 26:581-587, the teachings of which are incorporated herein; casein, gelatin, glutin, polyanhydrides, polyacrylic acid, alginate, chitosan, poly(methyl methacrylates), poly(ethyl methacrylates), poly(butylmethacrylate), polyhyaluronic acids, poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Other sustained release delivery systems useful according to the invention include, but are not limited to, fatty acids and a medicinal pump. Preferably the fatty acids are C₉-C₂₀ fatty acids.

The pharmaceutical compositions of the invention may be conveniently presented in unit dosage form and may be prepared by any of the methods well known in the art of pharmacy. Such methods include the step of bringing the pharmaceutical composition into association with a carrier which constitutes one or more accessory ingredients. In general, the compositions are prepared by uniformly and intimately bringing the pharmaceutical composition into association with a liquid carrier, a finely divided solid carrier, or both, and then, if necessary, shaping the product. The pharmaceutical composition may be stored in a lyophilized condition.

The pharmaceutical compositions can be suspended in a liquid, e.g., in dissolved form or colloidal form. The liquid can be a solvent, partial solvent, or non-solvent. In many cases, water or an organic liquid can be used.

The pharmaceutical compositions are administered to the subject in a therapeutically effective amount. The therapeutically-effective amount can be determined on an individual basis and is based, at least in part, on considerations of the age, sex, size, and health of the subject; the type of pharmaceutical composition used, the type of delivery system used; the time of administration; and whether a single, multiple, or controlled-release dose regimen is employed. A therapeutically-effective amount can be determined by one of ordinary skill in the art employing such factors and using no more than routine experimentation.

The dosage concentration of the pharmaceutical composition actually administered is dependent, at least in part, upon the final concentration of pharmaceutical composition that is desired at the site of action, the method of administration, the efficacy of the particular pharmaceutical composition, the longevity of the particular pharmaceutical composition, the weight or body mass index of the patient, and the timing of administration. Preferably, the dosage form is such that it does not substantially deleteriously affect the subject. The dosage can be determined by one of ordinary skill in the art employing such factors and using no more than routine experimentation.

The following examples serve to illustrate the invention without limiting it thereby. It will be understood that variations and modifications can be made without departing from the spirit and scope of the invention.

### EXAMPLE

Tissue specimens from normal parietal peritoneum and adhesions are collected from patients who are undergoing abdominal/pelvic surgical procedures.

After collection, the tissue pieces are divided into multiple portions, the fibroblasts are isolated, and then collected under Normoxic conditions. The expression of the molecular adhesion markers is determined by PCR, using the primers designated. Data are expressed as mean ±SEM, and the significance is defined as p<0.05. The data are statistically analyzed using a one way analysis of variance (ANOVA) approach and Dunn's multiple test.

Marker values are analyzed as normally distributed continuous variables using a three (3) factor ANOVA (analysis of variance) model: 3 levels of the Treatment factor (Control, DCA, NS); 2 levels of the Condition factor (Normoxia and Hypoxia); and 2 levels of the Cell Type Factor (Normal and Adhesion). The statistical analysis provides the significance of the three main effects and the interaction effects for these factors. The observed significance for Cell Type are adjusted for the main and interaction effects.

The parameters evaluated include the cell culture conditions, the cell type and the treatment conditions. The cell culture conditions are either Normoxia or Hypoxia (2% oxygen). The cell types are either normal peritoneum cells or adhesion cells. Six (6) cells are used for each evaluation, and the results are averaged. The results are summarized below for each marker.

Caspase 2

The Caspase 2 average value for normal peritoneal cells is 0.561±0.007, compared to 0.536±0.017 for adhesion cells.

Caspase 3

There are no significant effects of cell type (Normal 0.572±0.022 v. Adhesion 0.556±0.057) for the Caspase 3 marker.

Caspase 9

The average value for normal cells is 0.688±0.027, compared to 0.648±0.024 for adhesion cells. P=0.025 for the Caspase 9 marker.

PPARα

There are no significant effects of cell type (Normal 0.535±0.030 v. Adhesion 0.528±0.012) for the PPARα marker.

PPARβ

PPARβ levels are higher in normal fibroblast cells (0.462±0.011) v. adhesion fibroblast cells (0.445±0.009). P=0.074 for the PPARβ marker.

PPARγ1

PPARγ1 levels are significantly lower in normal fibroblast cells (0.608±0.023) as compared to adhesion fibroblast cells (0.661±0.033). P<0.001 for the PPARγ1 marker.

PPARγ2

The results show that there is a significant effect of treatment by comparing the normal fibroblast cells (0.412±0.056) with the adhesion fibroblast cells (0.374±0.020) for the PPARγ2 marker.

NF-Kappa B

NF-kappa B is present in significantly higher amounts (18%, p<0.01) in normal peritoneal fibroblast cells as compared to adhesion fibroblast cells, as determined by Western Blot analysis. Hypoxia decreased NF-kappa B levels by 10% (p<0.01) in adhesion fibroblast cells, and by 14% (p<0.01) in normal peritoneal fibroblast cells.

Each of the foregoing patents, patent applications and references that are recited in this application are herein incorporated in their entirety by reference. Having described the presently preferred embodiments, and in accordance with the present invention, it is believed that other modifications, variations and changes will be suggested to those skilled in the art in view of the teachings set forth herein. It is, therefore, to be understood that all such variations, modifications, and changes are believed to fall within the scope of the present invention as defined by the appended claims.

What is claimed is:

1. A method for the treatment of surgical adhesions, or the stimulation of fibrosis, comprises treating a patient at risk of developing adhesions with an effective amount of a therapeutic formulation containing a modulator of the activity of one or more molecular markers selected from the group consisting of Caspase 2, Caspase 3, Caspase 9, PPARα, PPARβ, PPARγ1, PPARγ2, NF-kappa B, HIF-1α, and mixtures thereof.

2. The method of claim 1 which comprises the treatment of surgical adhesions by inhibiting the activity of said molecular markers.

3. The method of claim 2 wherein the inhibitor is a competitive inhibitor which competes with the molecular marker for a binding site.

4. The method of claim 2 wherein the inhibitor is a non-competitive inhibitor which directly inhibits the molecule.

5. The method of claim 2 wherein the molecular marker is a member of the Caspase family selected form the subgroup consisting of Caspase 2, Caspase 3 and Caspase 9.

6. The method of claim 2 wherein the molecular marker is a member of the PPAR family selected form the subgroup consisting of PPARα, PPARβ, PPARγ1 and PPARγ2.

7. The method of claim 2 wherein the molecular marker is NF-kappa B.

8. The method of claim 2 wherein the inhibitor is an antibody.

9. The method of claim 2 wherein the inhibitor is an antisense molecule of the molecular marker that prevents translation of the molecular marker from its mRNA.

10. The method of claim 2 wherein the inhibitor is an antisense molecule that regulates the stability of the mRNA of the gene encoding the molecular marker.

11. The method of claim 2 wherein the inhibitor is an agent that binds to the promoter region of the gene encoding the molecular marker and prevents trans-acting elements from enhancing the transcription of the gene.

12. The method of claim 2 wherein the inhibitor is an agent that reduces the posttranslational modification of the molecular marker.

13. The method of claim 2 wherein the therapeutic formulation is locally administered at the site of potential adhesion formation.

14. The method of claim 1 which comprises the stimulation of fibrosis formation.

15. The method of claim 14 wherein the molecular marker is a member of the Caspase family selected form the subgroup consisting of Caspase 2, Caspase 3 and Caspase 9.

16. The method of claim 14 wherein the molecular marker is a member of the PPAR family selected form the subgroup consisting of PPARα, PPARβ, PPARγ1 and PPARγ2.

17. The method of claim 14 wherein the molecular marker is NF-kappa B.

18. The method of claim 14 wherein the treatment causes the adherence of selected tissues within the patient.

19. A pharmaceutical preparation for the treatment of surgical adhesions comprising the molecular marker modulator of claim 1, a pharmaceutically acceptable carrier, and an adjuvant.
